(19) United States
(12) Patent Application Publication
(10) Pub. No.: US 2008/0097079 A1
(43) Pub. Date: Apr. 24, 2008

(54) Cyclization of a Peptide

(76) Inventors: Francesca Quattrini, Naters (CH); Stephane Varray, Sierre (CH); Oleg Werbitzky, Veyras (CH); Thomas Zeiter, Visperterminen (CH)

Correspondence Address: NIXON & VANDERHYE, PC, 901 NORTH GLEBE ROAD, 11TH FLOOR, ARLINGTON, VA, 22203, US

(21) Appl. No.: 11/666,651

(22) PCT Filed: Oct. 24, 2005
(86) PCT No.: PCT/EP05/11383

§ 371 Date: Oct. 4, 2007

(30) Foreign Application Priority Data
Nov. 1, 2004 (EP) 04025866.7
Apr. 18, 2005 (EP) 05008380.7

(51) Int. Cl. C07K 7/00

(52) U.S. Cl. 530/345

(57) ABSTRACT

An improved method for cyclization of peptide (H)-D-Phe-Cys-Tyr-D-Trp-Lys-Val-Cys-Trp(NH2) by cystine formation is devised.

The present invention relates to an improved method for disulfid-bonding driven cyclization of a peptide.

Vapreotide is a cyclic octapeptide comprising one cystine moiety. It is a synthetic analogue of somatostatin which hormone inhibits secretion of hormones such as insulin and glucagon, important regulators of fat and sugar metabolism and growth hormone. Somatostatin got a physiological half-life of 3 minutes, whereas Vapreotide is much long-lasting in the circulation, owing to C-terminal amidation of the analogue. Disulfide bonding is a feature that is paramount for activity both of vapreotide and somatostatin.

Somatostatin has the structure:

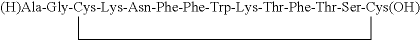

Vapreotide has the structure:

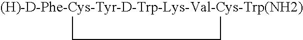

Volkmer-Engert et al. (Surface-assisted catalysis of intramolecular disulfide bond formation in peptides, J. Peptide Res. 51, 1998, 365-369) describe charcoal-catalyzed oxidative formation of intramolecular disulfide bonds in water by using oxygen dissolved in the solvent, i.e. water. Careful controls showed that the pool of oxygen physically dissolved in the aequeous medium was necessary and sufficient to load the charcoal with oxygen for oxidation. Use of charcoal, as compared to traditional air-sparging in the absence of catalyst, accelerated the reaction rate for intramolecular cystine formation dramatically and selectively.

The use of charcoal as a heterogenous catalyst inevitably requires to carry out such reaction in solution but not on-resin. It has become a widely applied method where applicable; the method imposes some restrictions on the solvent system not suitable for every peptide.

U.S. Pat. No. 6,476,186 devises intramolecular disulfide bonding of the linear, deprotected octapeptide D-Phe-Cys-Phe-D-Trp-Lys-Thr-Cys-Thr(OH) in acetonitril/water (1:1) in the presence of trace amounts of charcoal. The peptide was synthesized on 2-chlorotrityl resin and comprises apart from hydrophobic residues and the cysteines, a lysine and a threonine. Cysteines were protected with acid-labile trityl groups. Charcoal-catalyzed cyclization took place after cleavage and deprotection in the aequeous solvent mix, carefully adjusted to pH 8.0 with sodium hydroxide, for 5 hours at a concentration of 50 mg/mL. Yield of pure product was about 80%.

As a disadvantage, when seeking to apply the same methodology to non-cyclic Vapreotide which is a less water-soluble peptidamide in contrast, 80% analytical yield was achievable but only after 44 h of extended reaction time and at a dilution of about 1 mg/mL. Elevated concentrations of only 5-10 mg/mL gave rise to slow precipitation of educt, lowering the product yield accordingly. Higher permanent concentrations of about 10 mg/mL could be achieved by change in pH to pH 6, but ensued formation of a side product, hence reducing yield to 30%. For an efficient industrial-scale process, much shorter reaction times with higher through-put of material are needed.

It is an object of the present invention to devise another or improved method for cyclization of the linear peptide precursor of mature Vapreotide, avoiding the disadvantages of the prior art. This object is solved by the present method of cyclisizing a linear peptide (H)-D-Phe-Cys-Tyr-D-Trp-Lys-Val-Cys-Trp(NH2) by means of intramolecular cystine formation, comprising the step of cyclisizing said peptide at a concentration of at least 20 mg/mL in a polar, aprotic solvent and in the presence of charcoal in the further presence of a primary, secondary or tertiary amine as a base reagent and of an oxidizing reagent.

The afore said peptide may be produced by any method known in the art, be it biotechnologically or by means of liquid or solid phase chemical synthesis. Methods of solid-phase synthesis are described in Bodansky, M., Principles of Peptide Synthesis, 2^{nd} ed. Springer Verlag Berlin/Heidelberg, 1993, for instance. C-terminal amidation by may be achieved by enzymatic or chemical methods well-known in the art, e.g. by solid-phase synthesis on a resin in C-terminal linkage on well-known Sieber or Rink amide resin, giving rise to carboxamid upon cleavage from resin. Chemical synthesis usually requires use of protection groups for individual amino acids as is routinely known in the art. A large variety of protection groups can be employed for protection of cysteine residues, e.g. trityl, acetamidomethyl- (acm-), t-butyl, trimethylacetamidomethyl, 2,4,6-trimethoxybenzyl, methoxytrityl, t-butylsulphenyl. Removal of such protection groups will require specific reagents or reaction conditions, depending on the type of protection group employed.

Most commonly, the trityl group is employed for simple protection during peptide synthesis and may then be removed by simple acidolysis e.g. in 30-60% TFA concomittant with other protection groups and the resin linker. For the present invention, it is only needed that the cysteinylpeptide sought to be cyclizised has free, unprotected cysteines for applying the method of the present invention. As a side issue, it may be helpful to mention that Atherton et al. (1985, J. Chem. Perkin Trans. I., 2065) reported that upon acidolysis, the use of the popular scavenger thioanisol which is bifunctional in being both scavenger and also acidolysis promoter also resulted in partial deprotection of acm, tert-butyl and S-tert-butylsulphenyl protected cysteines. This would also allow of working the present invention. More quantitatively, S-tBu-sulphenyl groups are easily and selectively removable under mild reaction conditions by treatment with thiol regents such as beta-mercapto-ethanol or dithio-threitol, or by treatment with e.g. trisphenyl- or trisethyl-phosphine.

In contrast, customarily in the art acm-protection groups are quantitatively removed by iodine oxidation concomittant with cyclization; hence iodine oxidation-borne deprotection of acm groups is not compatible with the present invention.

The polar, aprotic solvent preferably is a solvent miscible with both water and aceton. Suitable examples of solvents of this type in accordance with the present invention are e.g. dimethyl-formamide (DMF), N-methyl-pyrrolidone (NMP), acetamide or tetrahydrofurane (THF). It goes without saying that a suitable solvent in the present context necessarily is compliant with achieving a peptide concentration of at least 20 mg/mL. Consequently, acetonitril is not a suitable solvent according to the present invention as is shown in the comparative examples in the experimental section. — Preferably, the solvent is selected from the group consisting of acetamide, dimethyl-formamide and N-methyl-pyrrolidone.

More preferably, the solvent is selected from the group consisting of dimethyl-formamide and N-methyl-pyrrolidone. Most preferably, the solvent is dimethyl-formamide.

Preferably, the base is a weak base of a conjugated pKa of from 7.5 to 10, and is a secondary, more preferably a sterically hindered, tertiary amine. Examples of such and further preferred are Hünig-base (N,N-diisopropylethylamine), N,N′-dialkylaniline, 2,4,6-trialkylpyridine or N-alkyl-morpholine with the alkyl being straight or branched C1-C4 alkyl, more preferably it is N-methylmorpholine or collidine (2,4,6-trimethylpyridine), most preferably it is collidine.

The oxidizing reagent preferably is air and/or oxygen. When switching from aequeous solvent system of the prior art to the non-aequeous solvent system of the present invention, it is strongly preferred to ensure saturation of the catalyst with oxygen by actively supplying or ventilating the solvent liquid with air and/or oxygen, at least during a period of time, e.g. by directing a steady or timewise stream of air from a submerged gas sparger into the solvent. Optionally, a strong vortex arising from stirring of the solution along with a favorable aspect ratio of the liquid phase relative to the vortex in order to avoid local mixing only, and with possibly suitably shaped, e.g. propeller-like or bead like, stirrer device or stirring means, can be used.

The charcoal or activated charcoal may be of the same type as employed in the prior art; preferably, it is used in at least catalytic amounts. It goes without saying that the reaction rate is both influenced by the amount of catalyst present and by the concentration of educts, the latter enhancing the absorption rate to or the loading of the solid surface of the heterogenous catalyst with cysteinyl-peptide from the solution. However, according to the present invention, the choice of solvent strongly contributes to efficient catalysis. Further it was observed, without wanting to be very strictly bound to such observation or any respective theory, that the reaction cinetics were depending not only on the solvent system u but also on the amount of educt: Whilst increasing the amount of educt in the range of 1 to 100 mg/mL, the reaction kinetics display an entirely surprising change in shape and become ever more linear rather than sigmoidal. Some yet unreported transition phenomenon at the phase border solid-liquid might account for that. In result, 100% conversion rates amounting to even somewhat improved product yields were achievable at dramatically shorter reaction times by increasing the educt concentration. This has not previously been accounted for when using charcoal-catalyzed oxidation. Therefore the afore cysteinylpeptide-precursor (H)-D-Phe-Cys-Tyr-D-Trp-Lys-Val-Cys-Trp(NH2), is added to the reaction in an amount of at least 20 mg/mL, more preferably 50 mg/mL, most preferably 100 mg/mL according to the present invention. The upper limit is given by the maximum solubility of course.

The cyclization reaction may be carried out under refluxing conditions at a temperature range of from about 0° C. to 80° C., more preferably of from 5° C. to 60° C., most preferably at about 15-40° C., and preferably in conjunction with using air/oxygen as oxidizing reagent.

### EXPERIMENTS

### 1. Synthesis of (H)-D-Phe-Cys-Tyr-D-Trp-Lys-Val-Cys-Trp(NH2)

Linear peptide was synthesized on Sieber-resin (from Novabiochem) by Fmoc standard methods. Side chain protection groups: D-oder L-Trp(Boc), Cys(Trt), Lys(Boc), Tyr(tBu). The protected peptide was cleaved off in 5% TFA in dichloremethane and then globally deprotected by acidolysis in a cleavage mix of (eq.=volume parts) 300 eq. conc. TFA, 12 eq. dithio threitol, 12. eq. dichloromethane, 50 eq. water (acqua dest.) at 1 h at room temperature. The product is precipitated by adding methyl-tbutyl-ether, complete removal of Boc groups in the product (H)-D-Phe-Cys-Tyr-D-Trp-Lys-Val-Cys-Trp(NH2) and purity was verified by HPLC.

All cyclisation reactions were carried out at 25° C. Vapreotide product of formula I 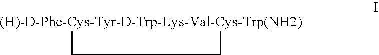
was always purified by directly loading the reaction mixture onto reverse phase HPLC.

### 2. Comparative Example: Cyclisation of (H)-D-Phe-Cys-Tyr-D-Trp-Lys-Val-Cys-Trp(NH2)

The product from exp. 1 was subjected to the charcoal method essentially as described in U.S. Pat. No. 6,476,186 and using acetonitril:water (1:1), except that a concentration of the linear cysteinylpeptide of 1 mg/mL was used, the pH was adjusted to about pH 8 with ammonia and that additionally air was sparged at low pressure into the liquid unter stirring. After 44 h, 100% conversion was achieved, with 84% (w/w) analytical yield after immediate HPLC purification of the product from the reaction mixture.—Higher concentrations of up to 10 mg/mL were previously found to be unstable and educt would have started to precipitate after a short time. Solubility of the peptide could also not be significantly improved by increasing the proportion of acetonitril:water to 3-2:1; rather reaction time at 1 mg/mL increased by 50-100%, with concomittant loss of purity (yield: 74% (w/w)).

### 3. Comparative Example: Cyclisation of (H)-D-Phe-Cys-Tyr-D-Trp-Lys-Val-Cys-Trp(NH2)

The reaction of exp. 2 was repeated, with the sole difference that ammonia/NH4Cl was used to adjust the pH to 6 and to dissolve the cysteinylpeptide at 10 mg/mL accordingly. After 70 hours, a conversion rate of 80% was reached whilst the product had only low purity. Yield of product I was 31%.

### 4. Cyclisation of (H)-D-Phe-Cys-Tyr-D-Trp-Lys-Val-Cys-Trp(NH2)

The product from exp. 1 was subjected to the charcoal method using trace amounts of activated, powdered charcoal, a concentration of the linear cysteinylpeptide of 50 mg/mL (1 eq.) in dimethylformamide in the presence of 1 eq. diisopryl-ethyl-amine and that additionally air was sparged at low pressure into the liquid unter stirring.

After 15-20 hours(!), 100% conversion was achieved with an analytical yield of product I of 79%. The experiment was conducted thrice independently, always with the same result.

1. Method for cyclization of a linear peptide (H)-D-Phe-Cys-Tyr-D-Trp-Lys-Val-Cys-Trp(NH2) by means of intramolecular cystine formation, comprising the step of cyclisizing said peptide at a concentration of at least 20 mg/mL in a polar, aprotic solvent selected from the group consisting of dimethyl-formamide, N-methyl-pyrrolidone, acetamide or tetrahydrofurane, and in the presence of charcoal in the further presence of a primary, secondary or tertiary amine as a base reagent and of an oxidizing reagent.

2. Method according to claim 1, characterized in that the charcoal is present in at least catalytic amount.

3. Method according to claim 1, characterized in that the oxidizing reagent is air and/or oxygen.

4. Method according to claim 3, characterized in that the reaction is supplied with air and/or oxygen during the reaction.

5. Method according to claim 4, characterized in that the reaction vessel has means to direct the air and/or oxygen directly into the liquid, preferably is comprising multiple orifices in the bottom and/or walls of a reaction vessel or is comprising at least one submerged gas sparger from which means or sparger air is bubbling directly into the solvent.

6. Method according to claim 1, characterized in that the solvent is selected from the group consisting of dimethylformamide and N-methyl-pyrrolidone.

7. Method according to claim 6, characterized in that the solvent is dimethylformamide.

8. Method according to claim 1, characterized in that the concentration of the peptide is at least 50 mg/mL, preferably that the solvent is dimethylformamide and that the concentration of the peptide is at least 100 mg/mL.

9. Method for cyclization of a linear peptide (H)-D-Phe-Cys-Tyr-D-Trp-Lys-Val-Cys-Trp(NH2) by means of intramolecular cystine formation, comprising the step of cyclisizing said peptide at a concentration of at least 20 mg/mL in a polar, aprotic solvent which is miscible both with water and acetone, and in the presence of charcoal in the further presence of a primary, secondary or tertiary amine as a base reagent and of an oxidizing reagent.
